(19) United States
(12) Patent Application Publication
(10) Pub. No.: US 2012/0226162 A1
(43) Pub. Date: Sep. 6, 2012

(54) ULTRASOUND PROBE AND ULTRASOUND DIAGNOSTIC APPARATUS

(75) Inventor: Katsuya YAMAMOTO, Ashigara-kami-gun (JP)

(73) Assignee: FUJIFILM CORPORATION, Tokyo (JP)

(21) Appl. No.: 13/398,020

(22) Filed: Feb. 16, 2012

(30) Foreign Application Priority Data
Mar. 3, 2011 (JP) 2011-046579

(51) Int. Cl. A61B 8/13

(52) U.S. Cl. 600/447

(57) ABSTRACT

An ultrasound probe includes a transducer array including vibrators arrayed in an azimuth direction each of which constitutes a single channel and has first transducer centrally located in an elevation direction and second transducers located on both sides of the first transducer, a first transmission and reception section for the first transducers of the respective channels, a second transmission and reception section for the second transducers of the respective channels, and a controller for controlling the first transmission and reception section to obtain reception data for B mode image through a simultaneous aperture for B mode image while controlling the first transmission and reception section and the second transmission and reception section to obtain reception data for sound speed through a simultaneous aperture for sound speed that is broader than the simultaneous aperture for B mode image.

### BACKGROUND OF THE INVENTION

The present invention relates to an ultrasound probe and an ultrasound diagnostic apparatus and particularly to an ultrasound probe and an ultrasound diagnostic apparatus for both producing a B mode image and measuring a sound speed.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In recent years, sound speeds in a region under examination are measured to achieve a more accurate diagnosis of a region inside the subject's body.

JP 2010-99452 A, for example, proposes an ultrasound diagnostic apparatus whereby a plurality of lattice points are set around a region under examination and an ultrasonic beam is transmitted and received for the lattice points to obtain reception data, based on which local sound speeds are calculated.

JP 2010-99452 A describes a device having an ultrasound probe that transmits and receives an ultrasonic beam to and from the inside of a subject's body to obtain local sound speeds at a region under examination, thereby enabling display of a B mode image with, for example, information on the local sound speeds superimposed over it. Further, producing a sound speed map representing a distribution of local sound speeds at respective points in a given region and displaying it together with the B mode image effectively support diagnosis of a region under examination.

In order to calculate a more accurate local sound speed, it is preferable to transmit ultrasonic beams so that well-focused transmission focuses are formed at respective lattice points set around a region to be diagnosed and receive ultrasonic echoes with a broad aperture as compared with when a B mode image is produced.

With a transducer array of an ultrasound probe, the transmission focuses of an ultrasonic beam can generally be formed at any depth desired by adjusting the amounts of delay between channels of vibrators arrayed in the azimuth direction, but the transmission focuses are often fixed focuses in the elevation direction whose positions are determined by an acoustic lens provided before the transducer array. Therefore, it is difficult to form a well-narrowed transmission focus at lattice points that are set at a depth other than that at which the fixed focuses are positioned by the acoustic lens, reducing the accuracy of sound speed measuring.

### SUMMARY OF THE INVENTION

An object of the present invention has been made to overcome such problems associated with the prior art and provide an ultrasound probe and an ultrasound diagnostic apparatus capable of both producing a B mode image and accurately measuring a sound speed.

An ultrasound probe according to the present invention comprises:

a transducer array including a plurality of vibrators arrayed in an azimuth direction of the transducer array, each vibrator constituting a single channel and having a first ultrasound transducer centrally located in an elevation direction of the transducer array and a pair of second ultrasound transducers located adjacent to and on both sides of the first ultrasound transducer;

a first transmission and reception section for performing transmission and reception of ultrasonic waves with the first ultrasound transducers of the respective channels;

a second transmission and reception section for performing transmission and reception of ultrasonic waves with the second ultrasound transducers of the respective channels; and

a controller for controlling the first transmission and reception section to obtain reception data for a B mode image by transmitting and receiving an ultrasonic beam for a B mode image through a simultaneous aperture for a B mode image formed by the first ultrasound transducers of given channels while controlling the first transmission and reception section and the second transmission and reception section to obtain reception data for measuring a sound speed by transmitting and receiving an ultrasonic beam for measuring a sound speed through a simultaneous aperture for measuring a sound speed that is broader than the simultaneous aperture for a B mode image.

An ultrasound diagnostic apparatus according to the present invention comprises the above ultrasound probe, an image producer for producing a B mode image based on the obtained reception data for a B mode image, and a sound speed calculator for calculating a sound speed based on the obtained reception data for measuring the sound speed.

### BRIEF DESCRIPTION OF THE DRAWINGS

FIG. 1 is a block diagram illustrating a configuration of an ultrasound diagnostic apparatus having an ultrasound probe according to Embodiment 1 of the invention.

FIG. 2 illustrates a configuration of a transducer array used in the ultrasound probe according to Embodiment 1.

FIGS. 3A and 3B schematically illustrate a principle of sound speed calculation in Embodiment 1.

FIGS. 4A, 4B, and 4C schematically illustrate a transmission and reception aperture for B mode image and a transmission and reception aperture for sound speed measurement in Embodiment 1.

FIG. 5 illustrates relationships between the positions of lattice points set for sound speed measurement and ultrasonic beams in Embodiment 1.

FIG. 6 illustrates relationships between the positions of lattice points set for sound speed measurement and ultrasonic beams in Embodiment 2.

### DETAILED DESCRIPTION OF THE INVENTION

Embodiments of the present invention will now be described below based on the appended drawings.

### Embodiment 1

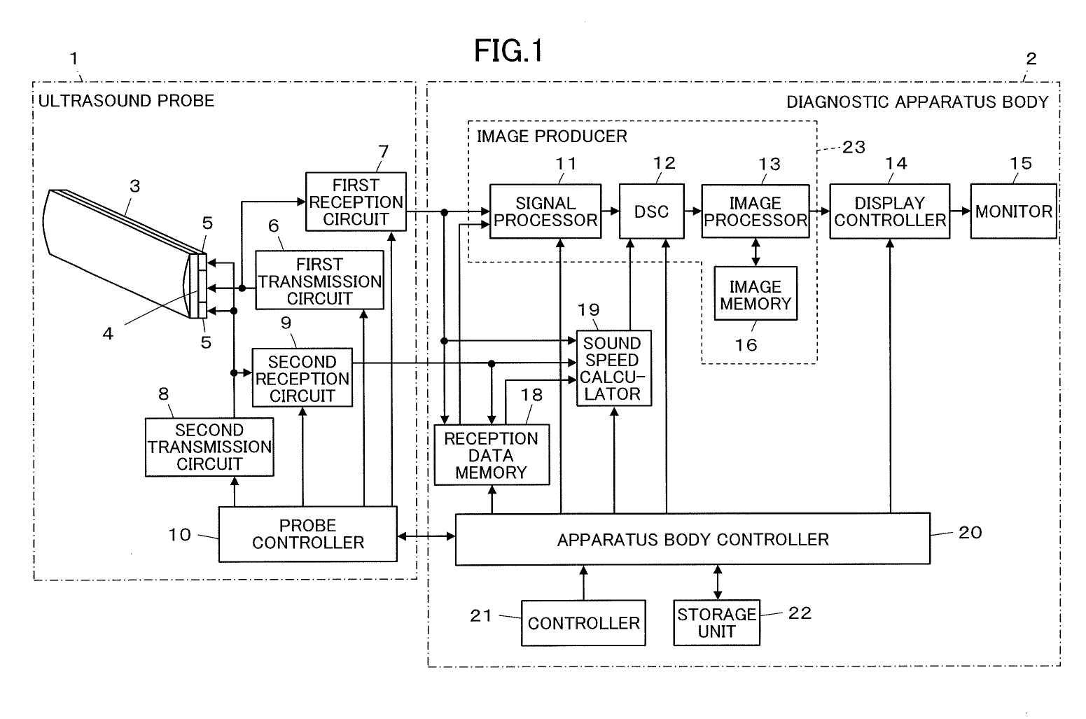

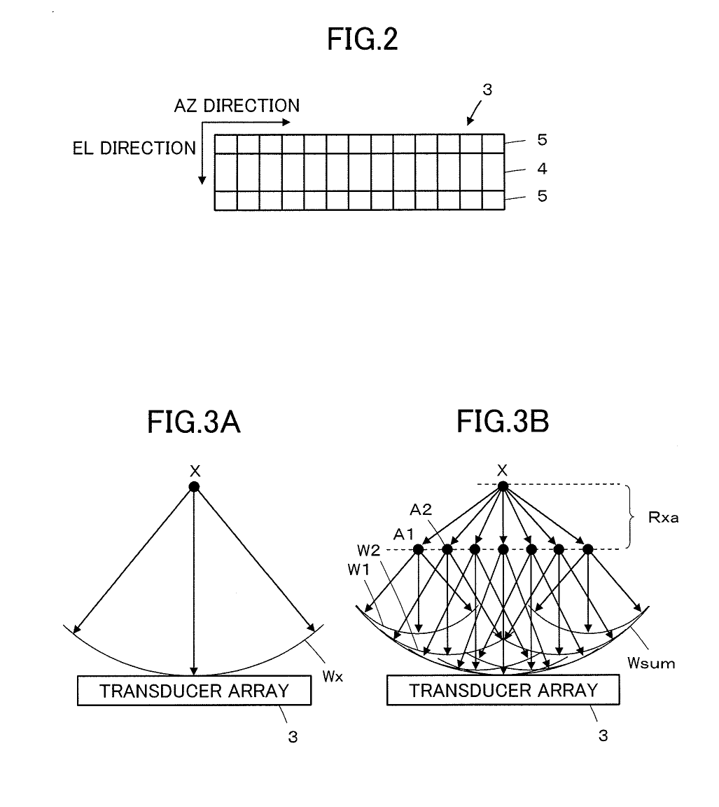

FIG. 1 illustrates a configuration of an ultrasound diagnostic apparatus having an ultrasound probe according to Embodiment 1 of the invention. The ultrasound probe 1 is connected to a diagnostic apparatus body 2.

The ultrasound probe 1 comprises a transducer array 3 for transmitting and receiving an ultrasonic beam. The transducer array 3 includes a plurality of channels of vibrators; the vibrator of each channel has a first ultrasound transducer 4 and a pair of second ultrasound transducers 5 located on both sides of the first ultrasound transducer 4.

The first ultrasound transducer 4 of each channel is connected to a first transmission circuit 6 and a first reception circuit 7; the second ultrasound transducers 5 of each channel are connected to a second transmission circuit 8 and a second reception circuit 9. The first transmission circuit 6, the first reception circuit 7, the second transmission circuit 8 and the second reception circuit 9 are connected to a probe controller 10.

A diagnostic apparatus body 2 comprises a signal processor 11 connected to the first reception circuit 7 of the ultrasound probe 1; the signal processor 11 is connected in sequence to a DSC (Digital Scan Converter) 12, an image processor 13, a display controller 14, and a monitor 15. The image processor 13 is connected to an image memory 16. The diagnostic apparatus body 2 further comprises a reception data memory 18 and a sound speed calculator 19 both connected to the first reception circuit 7 and the second reception circuit 9 of the ultrasound probe 1. The signal processor 11, the DSC 12, the display controller 14, the reception data memory 18, and the sound speed calculator 19 are connected to an apparatus body controller 20. The apparatus body controller 20 is connected to an operating unit 21 and a storage unit 22.

The probe controller 10 of the ultrasound probe 1 and the apparatus body controller 20 of the diagnostic apparatus body 2 are connected to each other.

The transducer array 3 includes a plurality of vibrators arrayed in azimuth direction; these vibrators constitute a plurality of channels. The vibrator of each channel is divided into three elements in the elevation direction. Specifically, each vibrator has the first ultrasound transducer 4 located centrally in the elevation direction and a pair of the second ultrasound transducers 5 located adjacent to and on both sides of the first ultrasound transducer 4 in the elevation direction.

The pair of the second ultrasound transducers 5 of each channel are connected to the second transmission circuit 8 and the second reception circuit 9 in common while the first ultrasound transducer 4 is connected to the first transmission circuit 6 and the first reception circuit 7, which are different from its counterparts to which the second ultrasound transducers 5 are connected. Thus, the first ultrasound transducer 4 and the second ultrasound transducers 5 can transmit and receive ultrasonic waves independently from each other.

The first ultrasound transducer 4 of each channel transmits ultrasonic waves for producing a B mode image or ultrasonic waves for measuring the sound speed according to a driving signal supplied from the first transmission circuit 6, receives ultrasonic echoes from a subject, and outputs a reception signal to the first reception circuit 7. The second ultrasound transducer 5 of each channel transmits ultrasonic waves for measuring a sound speed according to a driving signal supplied from the second transmission circuit 8, receives ultrasonic echoes from the subject, and outputs a reception signal to the second reception circuit 9.

Each of the first ultrasound transducers 4 and the second ultrasound transducers 5 comprises an element composed of a piezoelectric body and electrodes each provided on both ends of the piezoelectric body. The piezoelectric body is composed, for example, of a piezoelectric ceramic typified by a PZT (titanate zirconate lead), a polymeric piezoelectric device typified by PVDF (polyvinylidene flouride), or a piezoelectric monochristal typified by PMN-PT (lead magnesium niobate lead titanate solid solution).

When the electrodes of each of the ultrasound transducers are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce an electric signal, which is then outputted as reception signal of the ultrasonic waves.

The first transmission circuit 6 includes, for example, a plurality of pulsars and adjusts the amounts of delay for driving signals based on a transmission delay pattern selected according to a control signal transmitted from the probe controller 10 so that the ultrasonic waves transmitted from a plurality of the first ultrasound transducers 4 of the transducer array 3 form an ultrasonic beam, and supplies the first ultrasound transducers 4 with delay-adjusted driving signals.

The first reception circuit 7 amplifies and A/D-converts the reception signals transmitted from the first ultrasound transducers 4, and then performs reception focusing processing by providing the reception signals with respective delays according to the sound speed or sound speed distribution that is set based on a reception delay pattern selected according to the control signal transmitted from the probe controller 10 and adding up the reception signals. This reception focusing processing yields reception data (sound ray signals) having the ultrasonic echoes well focused.

Similarly to the first transmission circuit 6, the second transmission circuit 8 includes, for example, a plurality of pulsars and adjusts the amounts of delay for driving signals based on a transmission delay pattern selected according to a control signal transmitted from the probe controller 10 so that the ultrasonic waves transmitted from a plurality of the second ultrasound transducers 5 of the transducer array 3 form an ultrasonic beam, and supplies the second ultrasound transducers 5 with delay-adjusted driving signals.

The second reception circuit 9 amplifies and A/D-converts the reception signals transmitted from the second ultrasound transducers 5, and then performs reception focusing processing by providing the reception signals with respective delays according to the sound speed or sound speed distribution that is set based on a reception delay pattern selected according to the control signal transmitted from the probe controller 10 and adding up the reception signals to produce reception data (sound ray signal) where the ultrasonic echoes are well focused.

The probe controller 10 controls various components of the ultrasound probe 1 according to control signals transmitted from the apparatus body controller 20 of the diagnostic apparatus body 2.

The signal processor 11 of the diagnostic apparatus body 2 corrects attenuation in the reception data produced by the first reception circuit 7 of the ultrasound probe 1 according to the distance, i.e., the depth at which the ultrasonic waves are reflected, and then performs envelope detection processing to produce a B mode image signal, which is tomographic image information on a tissue inside the subject's body.

The DSC 12 converts the B mode image signal produced by the signal processor 11 into an image signal compatible with an ordinary television signal scanning mode (raster conversion).

The image processor 13 performs various processing required including gradation processing on the B mode image signal entered from the DSC 12 before outputting the B mode image signal to the display controller 14 or storing the B mode image signal in the image memory 16.

The signal processor 11, the DSC 12, the image processor 13, and the image memory 16 constitute an image producer 23.

The display controller 14 causes the monitor 15 to display an ultrasound diagnostic image based on the B mode image signal having undergone image processing by the image processor 13.

The monitor 15 includes a display device such as an LCD, for example, and displays an ultrasound diagnostic image under the control of the display controller 14.

The reception data memory 18 sequentially stores the reception data outputted from the first reception circuit 7 and the second reception circuit 9 of the ultrasound probe 1. The memory 18 stores information on a frame rate entered from the apparatus body controller 20 in association with the above reception data. Such frame rate information includes, for example, the depth of a position at which the ultrasonic waves are reflected, the density of scan lines, and a parameter representing the range of the visual field.

Under the control by the apparatus body controller 20, the sound speed calculator 19 calculates the local sound speeds for a tissue inside the subject's body under examination and also produces a sound speed map based on the reception data for measuring the sound speed among the reception data stored in the reception data memory 18.

The apparatus body controller 20 controls the components of the ultrasound diagnostic apparatus according to the instructions entered by the operator using the operating unit 21.

The operating unit 21 is provided for the operator to perform input operations and may be composed of, for example, a keyboard, a mouse, a track ball, and/or a touch panel.

The storage unit 22 stores, for example, an operation program and may be constituted by, for example, a recording medium such as a hard disk, a flexible disk, an MO, an MT, a RAM, a CD-ROM, a DVD-ROM, an SD card, a CF card, or a USB memory, a server, or the like.

The signal processor 11, the DSC 12, the image processor 13, the display controller 14, and the sound speed calculator 19 are each constituted by a CPU and an operation program for causing the CPU to perform various kinds of processing, but they may be each constituted by a digital circuit.

The operator may select one of the following three display modes using the operating unit 21. They are: a mode for displaying the B mode image alone; a mode for displaying the B mode image, with the sound speed map superimposed over the B mode image; and a mode for displaying the B mode image and the sound speed map in juxtaposition.

To display the B mode image, the first ultrasound transducers 4 of the transducer array 3 firstly transmit ultrasonic waves according to the driving signals supplied from the first transmission circuit 6 of the ultrasound probe 1, and the first ultrasound transducers 4 having received ultrasonic echoes from the subject output the reception signals to the first reception circuit 7, which produces the reception data. The signal processor 11 of the diagnostic apparatus body 2 having received the reception data produces the B mode image signal, and the DSC 12 performs raster conversion of the B mode image signal, while the image processor 13 performs various image processing on the B mode image signal, whereupon, based on this B-mode image signal, the display controller 14 causes the monitor 15 to display the ultrasound diagnostic image.

The local sound speed may be calculated by, for example, a method described in JP 2010-99452 A.

Suppose, as illustrated in FIG. 3A, that, on transmission of ultrasonic waves to the inside of a subject, reception waves Wx reach the transducer array 3 from the lattice point X, a reflection point in the subject, and that a plurality of lattice points A1, A2, . . . are arranged at equal intervals in positions shallower than the lattice point X, i.e., in positions closer to the transducer array 3, as illustrated in FIG. 3B. Then, the local sound speed at the lattice point X is obtained according to the Huygens principle whereby a synthesized wave Wsum produced by combining individual reception waves W1, W2, . . . transmitted from the lattice points A1, A2, . . . having received a reception signal from the lattice point X coincides with the reception waves Wx from the lattice point X.

First, an optimum sound speed for all the lattice points X, A1, A2, . . . is obtained. The optimum sound speed herein means a sound speed allowing a highest image contrast and sharpness to be obtained as a set sound speed is varied after performing focus calculation for the lattice points based on the set sound speeds and imaging to produce an ultrasound image. The optimum sound speed may be judged based on, for example, the image contrast, spatial frequency in the scan direction, and dispersion as described in JP 08-317926 A.

Next, the optimum sound speed for the lattice point X is used to calculate the waveform of an imaginary reception wave Wx emitted from the lattice point X.

Further, a hypothetical local sound speed V at the lattice point X is changed to various values to calculate the imaginary synthesized wave Wsum of the reception waves W1, W2, . . . from the lattice points A1, A2, . . . . Suppose that, at this time, the sound speed is consistent in a region Rxa between the lattice point X and the lattice points A1, A2, . . . and is equivalent to the local sound speed V at the lattice point X. The times in which the ultrasonic wave propagating from the lattice point X reaches the lattice points A1, A2, . . . are XA1/V, XA2/V, . . . , respectively, where XA1, XA2, . . . are the distances between the lattice point X and the lattice points A1, A2, . . . . Combining the reflected waves emitted from the lattice points A1, A2, . . . with respective delays corresponding to the times XA1/V, XA2/V, . . . yields the imaginary synthesized wave Wsum.

Next, the respective differences between a plurality of the imaginary synthesized waves Wsum calculated by changing the hypothetical local sound speed V at the lattice point X to various values and the imaginary reception waves Wx from the lattice point X are calculated to determine the hypothetical local sound speed V at which the difference becomes a minimum as the local sound speed. The difference between the imaginary synthesized waves Wsum and the imaginary reception waves Wx from the lattice point X may be calculated by any of appropriate methods including a method using the cross-correlation, a method using phase matching addition by multiplying the reception waves Wx by a delay obtained from the synthesized wave Wsum, and a method using phase matching addition by multiplying the synthesized wave Wsum by a delay obtained from the reception waves Wx.

Thus, the local sound speeds inside a subject can be accurately calculated based on the reception data for measuring the sound speed produced by the first reception circuit 7 and the second reception circuit 9 of the ultrasound probe 1. The sound speed map representing a distribution of the local sound speeds in a set region of interest may be likewise produced.

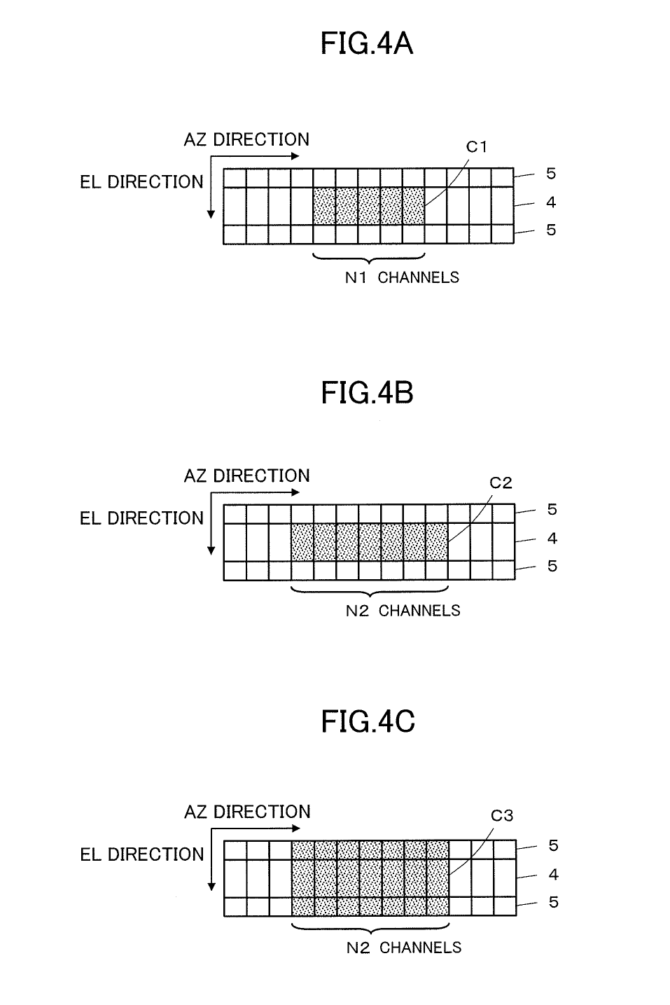

Referring now to FIG. 4, we will describe a simultaneous aperture for transmitting and receiving an ultrasonic beam for the B mode image and a simultaneous aperture for transmitting and receiving an ultrasonic beam for measuring the sound speed in the transducer array 3. Here, “a simultaneous aperture” means a region which is occupied with ultrasound transducers 4 simultaneously available for transmission and reception of an ultrasonic beam.

To transmit and receive an ultrasonic beam for the B mode image, the first ultrasound transducers 4 extending a given N1 number of channels first forms a simultaneous aperture C1 for the B mode image as illustrated in FIG. 4A.

To transmit and receive an ultrasonic beam for measuring the sound speed, two kinds of simultaneous apertures C2 and C3 are formed, both broader than the simultaneous aperture C1 for the B mode image, as follows.

The first simultaneous aperture C2 for measuring the sound speed is formed by the first ultrasound transducers 4 extending a given N2 number of channels, which is more than N1 channels of the simultaneous aperture C1 for the B mode image, so as to be longer in the azimuth direction than the simultaneous aperture C1 for the B mode image as illustrated in FIG. 4B. The second simultaneous aperture C3 for measuring the sound speed also extends the N2 number of channels similarly to the first simultaneous aperture C2 for measuring the sound speed and is formed by not only the first central ultrasound transducers 4 but the second ultrasound transducers 5 located on both sides thereof so as to be broader in the elevation direction than the first simultaneous aperture C2 for measuring the sound speed as illustrated in FIG. 4C

Next, the operation of Embodiment 1 will be described.

First, the first ultrasound transducers 4 extending a given N1 number of channels define the simultaneous aperture C1 for the B mode image as illustrated in FIG. 4A, whereupon the N1 number of channels of the first ultrasound transducers 4 contained in the simultaneous aperture C1 for the B mode image transmit and receive an ultrasonic beam for the B mode image according to the driving signals supplied from the first transmission circuit 6 and output reception signals to the first reception circuit, thereby producing reception data for the B mode image. The reception data are outputted to the reception data memory 18 and the image producer 23 of the diagnostic apparatus body 2, and the reception data memory 18 stores the reception data sequentially while the image producer 23 produces the B mode image signal. Based on the B mode image signal, the display controller 14 causes the monitor 15 to display the B mode image.

As the operator operates the operating unit 21 to set a region of interest R in the B mode image displayed on the monitor 15, the apparatus body controller 20 sets a plurality of lattice points for measuring the sound speed in the region of interest R and in its periphery.

Suppose, for example, that the region of interest R is set to extend over sound rays S6 to S8 among sound rays S1 to S13 formed at intervals corresponding to those at which a plurality of ultrasound transducers of the transducer array 3 are arrayed and at a depth ranging from L1 to L2 as illustrated in FIG. 5, whereas, for the region of interest R, nine lattice points E1 are set on sound rays S3 to S11 at the depth L1 along the upper edge of the region of interest R while three lattice points E2 are set on sound rays S6 to S8 at the depth L2 along the lower edge of the region of interest R. In FIG. 5, the lattice points E1 and E2 are indicated by “.”

First, an ultrasonic beam for measuring the sound speed is transmitted and received so that the transmission focus is formed at each of the nine lattice points E1.

Specifically, the first ultrasound transducers 4 and the second ultrasound transducers 5 extending the given N2 number of channels as illustrated in FIG. 4C define the second simultaneous aperture C3 for measuring the sound speed, whereupon the N2 number of channels of the first ultrasound transducers 4 contained in the second simultaneous aperture C3 for measuring the sound speed transmit ultrasonic waves according to the driving signals supplied from the first transmission circuit 6 whereas the N2 number of channels of the second ultrasound transducers 5 contained in the second simultaneous aperture C3 for measuring the sound speed also transmit ultrasonic waves according to the driving signals supplied from the second transmission circuit 8.

Now, the probe controller 10 controls the first transmission circuit 6 and the second transmission circuit 8 to set a given amount f delay between the ultrasonic waves transmitted from the first ultrasound transducers 4 and the ultrasonic waves transmitted from the second ultrasound transducers 5 of the respective channels, so that an ultrasonic beam B31 for measuring the sound speed that forms a transmission focus narrowed in the elevation direction at each of the lattice points E1 at the depth L1 is formed as illustrated in FIG. 5.

The ultrasonic beam B31 for measuring the sound speed having the transmission focuses at the nine lattice points E1 at the depth L1 is transmitted sequentially, and the reception signals are outputted to the first reception circuit 7 from the first ultrasound transducers 4 contained in the second simultaneous aperture C3 for measuring the sound speed having received ultrasonic echoes from the subject while the reception signals are outputted to the second reception circuit 9 from the second ultrasound transducers 5 contained in the second simultaneous aperture C3 for measuring the sound speed.

Thus, the first reception circuit 7 and the second reception circuit 9 having received the reception signals from the first ultrasound transducers 4 and the second ultrasound transducers 5 produce reception data for measuring the sound speed, which are stored in the reception data memory 18.

Subsequently to transmission and reception of the ultrasonic beams for measuring the sound speed for the nine lattice points E1, the ultrasonic beams for measuring the sound speed are transmitted and received so as to form the transmission focus at the three lattice points E2 at the depth L2.

In this case also, the first ultrasound transducers 4 and the second ultrasound transducers 5 extending over the given N2 number of channels illustrated in FIG. 4C define the second simultaneous aperture C3 for measuring the sound speed and transmit ultrasonic waves. The probe controller 10 controls the first transmission circuit 6 and the second transmission circuit 8 to change the amounts of delay set between the ultrasonic waves transmitted from the first ultrasound transducers 4 and the ultrasonic waves transmitted from the second ultrasound transducers 5 of the respective channels, so that an ultrasonic beam B32 for measuring the sound speed that forms the transmission focus narrowed in the elevation direction at each of the lattice points E2 at the depth L2 is formed as illustrated in FIG. 5.

The ultrasonic beam B32 for measuring the sound speed that forms the transmission focus at the nine lattice points E2 at the depth L2 is sequentially transmitted, and the reception signals are outputted to the first reception circuit 7 from the first ultrasound transducers 4 contained in the second simultaneous aperture C3 for measuring the sound speed having received ultrasonic echoes from the subject while the reception signals are outputted to the second reception circuit 9 from the second ultrasound transducers 5 contained in the second simultaneous aperture C3 for measuring the sound speed to produce the reception data for measuring the sound speed, which are stored in the reception data memory 18.

When the reception data for measuring the sound speed have been thus acquired for all the lattice points E1 and E2, the apparatus body controller 20 instructs the sound speed calculator 19 to calculate the sound speed, whereupon the sound speed calculator 19 calculates the local sound speeds in the region of interest R using the reception data for measuring the sound speed from among the reception data stored in the reception data memory 18.

Further, the sound speed calculator 19 produces the sound speed map for the region of interest R based on the local sound speeds at a plurality of points in the region of interest R, and data on the sound speed map undergo raster conversion through the DSC 12 as well as various image processing through the image processor 13 before being transmitted to the display controller 14. Then, the monitor 15 displays the B mode image, with the sound speed map superimposed over it, or the B mode image and the sound speed map in juxtaposition, depending on the display mode entered from the operating unit 21 by the operator.

Production of the B mode image, calculation of the local sound speeds, and production of the sound speed map are thus achieved.

Because the second simultaneous aperture C3 for measuring the sound speed is broader in the azimuth and elevation directions than the simultaneous aperture C1 for the B mode image, and the delay between the ultrasonic waves transmitted from the first ultrasound transducers 4 and the ultrasonic waves transmitted from the second ultrasound transducers 5 contained in the channels of the second simultaneous aperture C3 for measuring the sound speed is adjusted according to the depths of the lattice points E1 and E2, the ultrasonic beams B31 and B32 for measuring the sound speed can be formed with the transmission focuses narrowed at the lattice points E1 and E2 respectively, so that a high-accuracy sound speed measuring is made possible.

### Embodiment 2

In Embodiment 1, the ultrasonic beams B31 and B32 for measuring the sound speed are transmitted and received for the lattice points E1 and E2 at the depths L1 and L2 through the second simultaneous aperture C3 for measuring the sound speed. However, when it is preferable, given lattice points set at several depths, to form the transmission focuses of the ultrasonic beam for measuring the sound speed in a broad range, not only the second simultaneous aperture C3 for measuring the sound speed but, depending on the depth, the first simultaneous aperture C2 for measuring the sound speed extending, as illustrated in FIG. 4B, longer in the azimuth direction than the simultaneous aperture C1 for the B mode image may also be used.

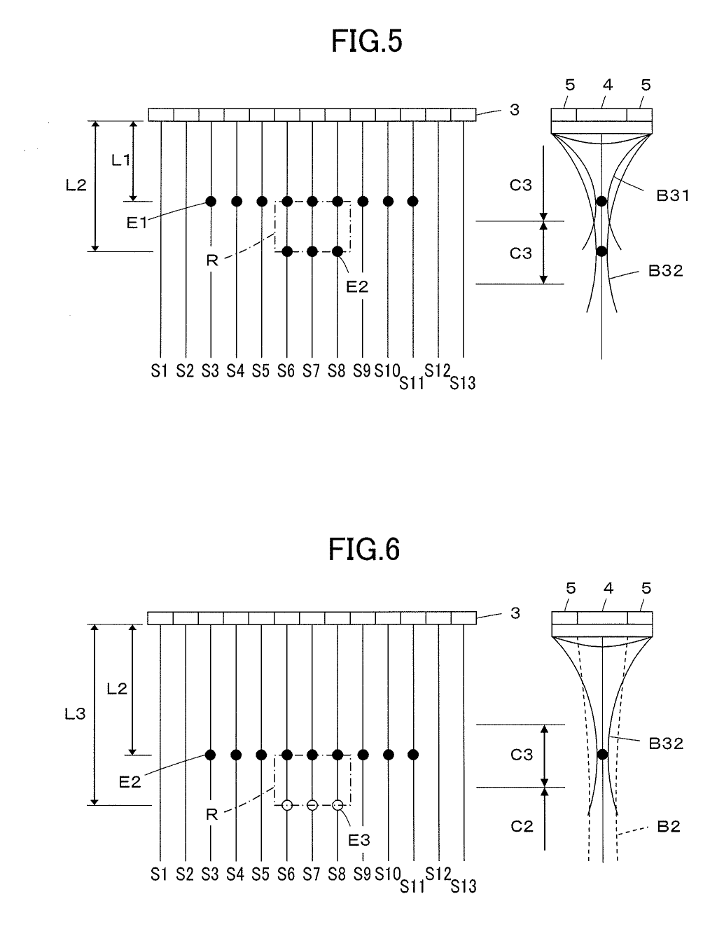

When, for example, the region of interest R having a depth ranging from L2 to L3 is set and, for this region of interest R, nine lattice points E2 are set at the depth L2 corresponding to the upper edge of the region of interest R and on the sound rays S3 to S11 while three lattice points E3 indicated by “0” are set at the depth L3 corresponding to the lower edge of the region of interest R and on the sound rays S6 to S8 as illustrated in FIG. 6, the ultrasonic beam B32 for measuring the sound speed is transmitted and received through the second simultaneous aperture C3 for measuring the sound speed for the lattice points E2 at the depth L2 as in Embodiment 1, whereas for the lattice points E3 located further deeper at the depth L3, the ultrasonic beam B2 for measuring the sound speed is transmitted and received through the first simultaneous aperture C2.

Thus, the ultrasonic beam B32 for measuring the sound speed that forms the transmission focus narrowed at each of the lattice points E2 at the depth L2 in the elevation direction is formed as illustrated in FIG. 6 by setting the second simultaneous aperture C3 for measuring the sound speed illustrated in FIG. 4C for the lattice points E2 at the depth L2 and a delay between the ultrasonic waves transmitted from the first ultrasound transducers 4 and the ultrasonic waves transmitted from the second ultrasound transducers 5 contained in the second simultaneous aperture C3 for measuring the sound speed.

The ultrasonic beam B32 for measuring the sound speed forming the transmission focus at the nine lattice points E2 at the depth L2 is sequentially transmitted, and the reception signals are outputted to the first reception circuit 7 from the first ultrasound transducers 4 contained in the second simultaneous aperture C3 for measuring the sound speed having received ultrasonic echoes from the subject while the reception signals are outputted to the second reception circuit 9 from the second ultrasound transducers 5 contained in the second simultaneous aperture C3 for measuring the sound speed to produce reception data for measuring the sound speed, which are stored in the reception data memory 18.

Next, the first simultaneous aperture C2 for measuring the sound speed illustrated in FIG. 4B is set for the lattice points E3 at the depth L3. The ultrasonic beam B2 for measuring the sound speed forming the transmission focus at the lattice points E3 at the depth L2 is transmitted from the first ultrasound transducers 4 of the N2 number of channels contained in the first simultaneous aperture C2 for measuring the sound speed.

Because the ultrasonic beam B2 for measuring the sound speed is formed only by the first ultrasound transducers 4 without using the second ultrasound transducers 5, the transmission focuses are narrowed to a smaller degree in the elevation direction, but the beam is focused over a wide range in the depth direction as compared with the ultrasonic beam B32 for measuring the sound speed formed through the second simultaneous aperture C3 for measuring the sound speed. Therefore, the ultrasonic beam B2 is effective for forming the transmission focuses at various depths.

The ultrasonic beam B2 for measuring the sound speed that forms the transmission focus at the three lattice points E3 at the depth L3 is sequentially transmitted, and the reception signals are outputted to the first reception circuit 7 from the first ultrasound transducers 4 contained in the first simultaneous aperture C2 for measuring the sound speed having received ultrasonic echoes from the subject to produce the reception data for measuring the sound speed, which are stored in the reception data memory 18.

Thus, upon acquisition of the reception data for measuring the sound speed for all the lattice points E2 and E3, the sound speed calculator 19 produces the local sound speeds in and the sound speed map for the region of interest R, whereupon the monitor 15 displays the B mode image with the sound speed map superimposed thereon or the B mode image and the sound speed map arranged in juxtaposition with each other depending on the display mode entered by the operator using the operating unit 21.

While, in Embodiments 1 and 2, the reception data outputted from the first reception circuit 7 and the second reception circuit 9 are provisionally stored in the reception data memory 18, and the sound speed calculator 19 uses the reception data stored in the reception data memory 18 to calculate the local sound speeds, the sound speed map producer 19 may directly receive the reception data outputted from the first reception circuit 7 and the second reception circuit 9 to calculate the local sound speeds.

Because the reception data memory 18 stores not only the reception data used to measure the sound speed but the reception data for the B mode image, the reception data for the B mode image may be read from the reception data memory 18 as necessary according to the control given by the apparatus body controller 20 for the image producer 23 to produce the B mode image.

The connection between the ultrasound probe 1 and the diagnostic apparatus body 2 may be achieved according to Embodiments 1 and 2 by wired communication or wireless communication.

What is claimed is:

1. An ultrasound probe for transmitting an ultrasonic beam toward a subject and receives ultrasonic echoes from the subject, comprising:
a transducer array including a plurality of vibrators arrayed in an azimuth direction of the transducer array, each vibrator constituting a single channel and having a first ultrasound transducer centrally located in an elevation direction of the transducer array and a pair of second ultrasound transducers located adjacent to and on both sides of the first ultrasound transducer;
a first transmission and reception section for performing transmission and reception of ultrasonic waves with the first ultrasound transducers of the respective channels;
a second transmission and reception section for performing transmission and reception of ultrasonic waves with the second ultrasound transducers of the respective channels; and
a controller for controlling the first transmission and reception section to obtain reception data for a B mode image by transmitting and receiving an ultrasonic beam for a B mode image through a simultaneous aperture for a B mode image formed by the first ultrasound transducers of given channels while controlling the first transmission and reception section and the second transmission and reception section to obtain reception data for measuring a sound speed by transmitting and receiving an ultrasonic beam for measuring a sound speed through a simultaneous aperture for measuring a sound speed that is broader than the simultaneous aperture for a B mode image.

2. The ultrasound probe according to claim 1,
wherein the first transmission and reception section includes a first transmission circuit for transmitting ultrasonic waves from the first ultrasound transducers of the respective channels and a first reception circuit for receiving ultrasonic waves to the first ultrasound transducers of the respective channels, and
wherein the second transmission and reception section includes a second transmission circuit for transmitting ultrasonic waves from the second ultrasound transducers of the respective channels and a second reception circuit for receiving ultrasonic waves to the second ultrasound transducers of the respective channels.

3. The ultrasound probe according to claim 2, wherein the controller controls the first transmission circuit and the second transmission circuit to adjust positions of transmission focuses of the ultrasonic beams for measuring a sound speed in a depth direction by setting a delay between ultrasonic waves transmitted from the first ultrasound transducers and ultrasonic waves transmitted from the second ultrasound transducers of the respective channels.

4. The ultrasound probe according to claim 2, wherein the controller controls the first transmission circuit and the first reception circuit so that the simultaneous aperture for measuring a sound speed is broader in the azimuth direction than the simultaneous aperture for a B mode image.

5. The ultrasound probe according to claim 4, wherein the controller controls the first transmission circuit, the second transmission circuit, the first reception circuit and the second reception circuit so that the simultaneous aperture for measuring a sound speed is broader in the elevation direction than the simultaneous aperture for a B mode image.

6. The ultrasound probe according to claim 3, wherein the controller controls the first transmission circuit and the first reception circuit so that the simultaneous aperture for measuring a sound speed is broader in the azimuth direction than the simultaneous aperture for a B mode image.

7. The ultrasound probe according to claim 6, wherein the controller controls the first transmission circuit, the second transmission circuit, the first reception circuit and the second reception circuit so that the simultaneous aperture for measuring a sound speed is broader in the elevation direction than the simultaneous aperture for a B mode image.

8. An ultrasound diagnostic apparatus comprising:
the ultrasound probe of claim 1;
an image producer for producing a B mode image based on the obtained reception data for a B mode image; and
a sound speed calculator for calculating a sound speed based on the obtained reception data for measuring a sound speed.
